(19) United States
(12) Patent Application Publication
(10) Pub. No.: US 2022/0379065 A1
(43) Pub. Date: Dec. 1, 2022

(54) NO KINK OXYGEN HOSE

(71) Applicant: Matthew H. Quintana, Pecos, NM (US)

(72) Inventor: Matthew H. Quintana, Pecos, NM (US)

(21) Appl. No.: 17/335,055

(22) Filed: May 31, 2021

(51) Int. Cl. A61M 16/08; F16L 11/08

(52) U.S. Cl. CPC A61M 16/0875; F16L 11/08; A61M 16/0816; A61M 2202/0208

(57) ABSTRACT

An illustrated view of a non-kinking air hose for preventing an air hose from kinking is presented. The non-kinking air hose is useful for providing an air flow from an air tank to a patient where the air hose is prevented from forming a kinking condition. The non-kinking air hose further prevents damage or reduced/zero air flow from an air tank to a patient.

### FIELD OF THE INVENTION

This invention relates to oxygen therapy. More particularly, it relates to a device to prevent hosing from kinking during oxygen therapy.

### BACKGROUND

Oxygen therapy, also known as supplemental oxygen, is the use of oxygen as a medical treatment. This can include for low blood oxygen, carbon monoxide toxicity, duster headaches, and to maintain enough oxygen while inhaled anesthetics are given. Long-term oxygen is often useful in people with chronically low oxygen such as from severe COPD or cystic fibrosis. Oxygen can be given in a number of ways including nasal cannula, face mask, and inside a hyperbaric chamber.

Oxygen is required for normal cell metabolism. Excessively high concentrations can cause oxygen toxicity such as lung damage or result in respiratory failure in those who are predisposed. Higher oxygen concentrations also increase the risk of fires, particularly while smoking, and without humidification can also dry out the nose. The target oxygen saturation recommended depends on the condition being treated. In most conditions a saturation of 94-96% is recommended, while in those at risk of carbon dioxide retention saturations of 88-92% are preferred, and in those with carbon monoxide toxicity or cardiac arrest they should be as high as possible. Air is typically 21% oxygen by volume while oxygen therapy increases this by some amount up to 100%.

When a patient requires the use of oxygen, whether in the hospital, home, etc., the patient moves around to further help in their recovery. One of the main issues related to the patient moving while connected to an oxygen tank is that an air hose connected to the oxygen tank and the patient often becomes tangled and can cause a crinkling in the air hose. This can cause damage to the air hose or a reduction or failure to provide oxygen from the oxygen tank to the patient.

In light of the foregoing, it would be desirable to devise a device that can prevent the crinkling of the air hose. It would be further advantageous if the device were easy to use and was cost efficient.

### BRIEF DESCRIPTION OF THE DRAWINGS

FIG. 1 is an illustrated view of an air hose with an exemplary non-kinking air hose.

FIG. 2 is an illustrated use of the non-kinking air hose shown in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

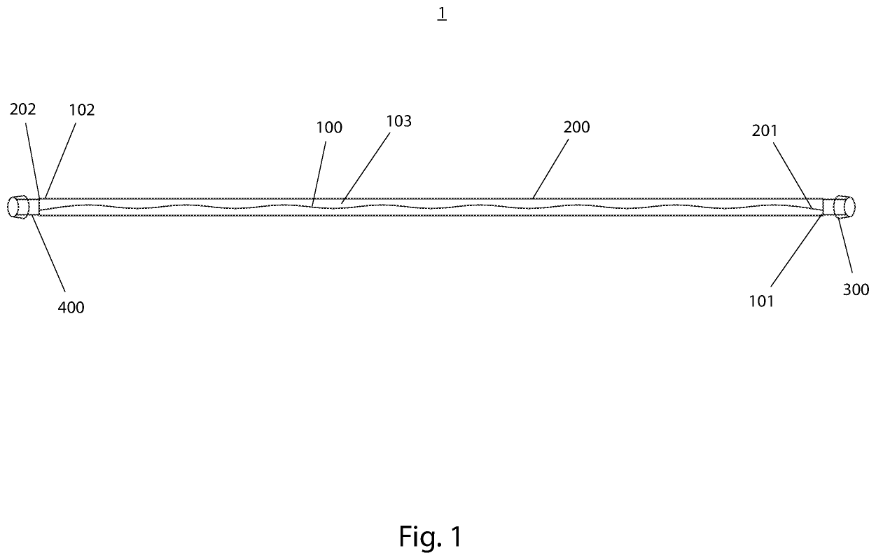

Referring to FIG. 1, an illustrated view of a non-kinking air hose 1 for preventing the air hose from kinking is presented. The non-kinking air hose 1 is useful for providing an air flow from an air tank to a patient where the air hose is prevented from forming a kinking condition. The non-kinking air hose 1 further prevents damage or reduced/zero air flow from an air tank to a patient.

The non-kinking air hose 1 has a non-kinking wire 100 and an air hose 200. The non-kinking wire 100 has a first end 101 and a second end 102.

The non-kinking wire 100 is made of a hardened monofilament line made in an extrusion process. The non-kinking wire 100 is preferably made of a nylon material, however other materials are hereby contemplated, including, but not limited to, metal, poly-vinyl chloride (PVC), high-density polyethylene (HDPE), low-density polyethylene (LDPE), etc. The non-kinking wire 100 preferably has a length of twenty-four (24) feet, however other lengths are hereby contemplated, including, but not limited to, nine (9) feet, forty-nine (49) feet, etc. The non-kinking wire 100 is preferably no less than one (1) foot less than a length of the air hose 200. The non-kinking wire 100 is preferably a diameter of ninety-eight thousandths (0.098) millimeter, however other diameters are hereby contemplated, including, but not limited to, seventy-five thousandths (0.075) millimeter, one-hundred ten thousandths (0.110) millimeters, etc.

The air hose 200 preferably has a length of twenty-five (25) feet, however, other lengths are hereby contemplated, including, but not limited to, ten (10) feet, fifty (50) feet, etc. The air hose 200 is preferably made of a high-density polyethylene (HDPE) material, however other materials are hereby contemplated, including, but not limited to, low-density polyethylene (LDPE), vinyl, plastic, etc.

The air hose 200 has a first end 201, a second end 202 and a middle 203. The middle 203 of the air hose 200 is preferably hollow such that the oxygen from an oxygen tank is allowed to flow from the first end 201 through the middle 203 then dispersed at the second end 202.

The first end 101 of the non-kinking wire 100 is inserted into the middle 203 of the air hose 200 by a vacuum pressure process.

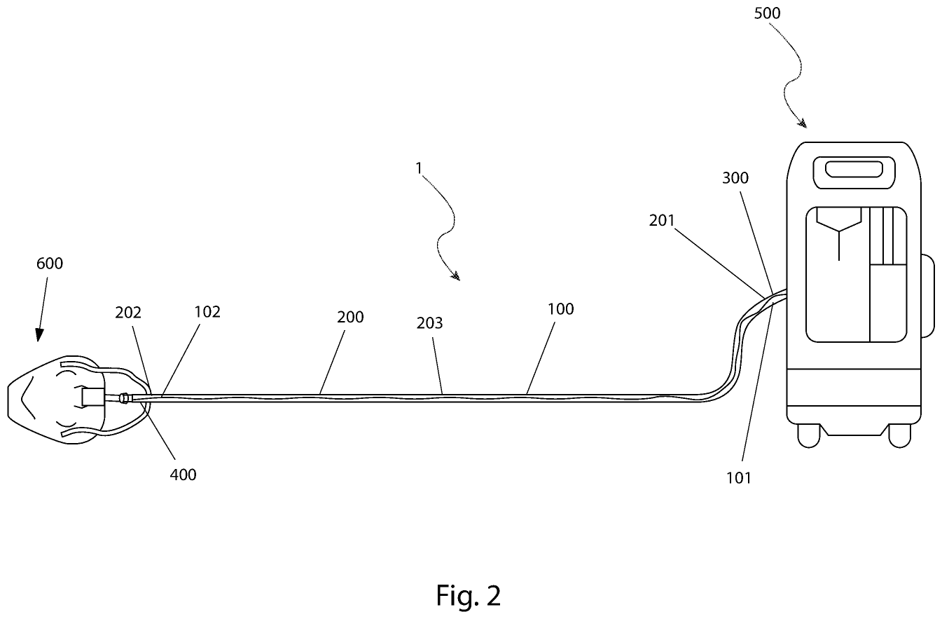

The first connector 300 is then securely coupled to the first end 201 of the air hose 200. The second end 202 of the air hose 200 is then securely coupled to the second connector 400. The first connector 300 is securely and removably coupled to an oxygen tank 500 of FIG. 2. The second connector 400 is coupled to a patient oxygen device for the patient to receive the flow of oxygen from the oxygen tank 500 of FIG. 2. The patient oxygen receiving device 600 of FIG. 2 is preferably a mask or nasal dispensing device, etc.

Moving now to FIG. 2, an illustrated use of the non-kinking air hose 1 shown in FIG. 1 is presented.

The non-kinking air hose 1 has a non-kinking wire 100 and an air hose 200. The non-kinking wire 100 has a first end 101 and a second end 102.

An oxygen tank 500 produces oxygen and pressures the oxygen to be delivered through an air hose 200 to a patient (not shown).

The first end 101 of the non-kinking wire 100 is placed into the middle portion 203 of the air hose 200 by a vacuum process. A first connector 300 is securely and removably coupled to the first end 201 of the air hose 200. The first connector 300 is configured to be coupled to the oxygen tank 500.

A second connector 400 is securely and removably coupled to the second end 202 of the air hose 200. The second connector 400 is configured to be coupled to a patient oxygen receiving device 600, such as a mask, air tube, etc., such that the patient may receive an air flow produced by the oxygen tank 500 and the air flow flowing through the air hose 200 to the patent.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A non-kinking air hose for preventing the air hose from kinking, the non-kinking air hose comprising:
a non-kinking wire, the non-kinking wire having a first end and a second end;
an air hose, the air hose having a first end, a second end and a middle portion, wherein the first end of the non-kinking wire is configured to be inserted into the middle portion of the air hose by a vacuum process;
a first connector, the first connector being coupled to the first end of the air hose, wherein the first connector being configured to be removably coupled to an air tank; and
a second connector, the second connector being coupled to the second end of the air hose, wherein the second connector being configured to be removably coupled to a patient oxygen device, wherein a flow of oxygen being received by the patient through a patient oxygen receiving device from the air tank.

2. The non-kinking air hose according to claim 1, wherein the non-kinking wire being a hardened monofilament line.

3. The non-kinking air hose according to claim 2, wherein the hardened monofilament line being made by an extrusion process.

4. The non-kinking air hose according to claim 1, wherein the non-kinking wire being made of a nylon material.

5. The non-kinking air hose according to claim 1, wherein the non-kinking wire having a length of twenty-four (24) feet.

6. The non-kinking air hose according to claim 1, wherein the non-kinking wire having a diameter being

7. The non-kinking air hose according to claim 1, wherein the non-kinking wire having a length no less than one (1) foot of a length of the air hose.

8. The non-kinking air hose according to claim 1, wherein the air hose having a length being twenty-five (25) feet.

9. The non-kinking air hose according to claim 1, wherein the air hose being made of a high-density polyethylene (HDPE) material.
